(19) United States
(12) Patent Application Publication
(10) Pub. No.: US 2008/0194575 A1
(43) Pub. Date: Aug. 14, 2008

(54) Treatment for non-alcoholic-steatohepatitis

(76) Inventors: Naiara Beraza, Aachen (DE); Michel Dreano, Saleve (FR); Christian Trautwein, Aachen (DE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/906,328

(22) Filed: Oct. 1, 2007

Related U.S. Application Data
(60) Provisional application No. 60/849,251, filed on Oct. 4, 2006.
(60) Provisional application No. 60/904,116, filed on Feb. 28, 2007.

(51) Int. Cl. A61K 31/496; A61P 3/04; A61P 3/08; A61P 3/06

(52) U.S. Cl. 514/252.14

(57) ABSTRACT

The present invention provides methods of treating a subject with non-alcoholic fatty liver disease (NAFLD), insulin resistance, obesity or hyperlipidemia, comprising administering to the subject an effective amount of a compound according to Formula I:

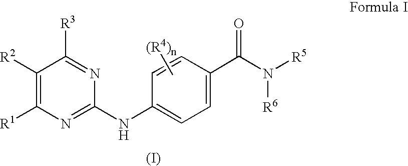

or a physiologically acceptable salt thereof.

### RELATED APPLICATIONS

This application claims the benefit of U.S. Provisional Applications 60/849,251, filed Oct. 4, 2006 and U.S. Provsional Application No. 60/904,116, filed Feb. 28, 2007. The entire teachings of these applications are incorporated herein by reference.

### BACKGROUND OF THE INVENTION

Nonalcoholic fatty liver disease (NAFLD) refers to a wide spectrum of liver disease ranging from simple fatty liver (steatosis), to nonalcoholic steatohepatitis (NASH), to cirrhosis (irreversible, advanced scarring of the liver). All of the stages of NAFLD have in common the accumulation of fat (fatty infiltration) in the liver cells (hepatocytes). In NASH, the fat accumulation is associated with varying degrees of inflammation and scarring of the liver.

NASH is more common in women and the most common cause is obesity. It is also often accompanied by visceral fat distribution, insulin resistance, dyslipidemia and hypertension. NASH can progress to fibrosing, steatohepatitis and trigger cirrhosis, end-stage liver disease and hepatocellular carcinoma. NASH is becoming recognized as the most common cause of liver disease, second only to Hepatitis C in numbers of patients going on to cirrhosis.

There are currently no specific therapies for NASH/NAFLD. The most important recommendations given to persons with this disease are to reduce their weight (if obese or overweight), follow a balanced and healthy diet, increase physical activity, avoid alcohol, and avoid unnecessary medications. Experimental approaches under evaluation in patients with NASH include antioxidants, such as vitamin E, selenium, and betaine. Another experimental approach to treating NASH is the use of newer antidiabetic medications—even in persons without diabetes. However, the effectiveness of these drugs is unknown. Thus, a need exists for new treatments for NASH.

### SUMMARY OF THE INVENTION

The present invention provides a method of treating nonalcoholic fatty liver disease (NAFLD) including fatty liver (steatosis), nonalcoholic steatohepatitis (NASH), and cirrhosis (advanced scarring of the liver).

The method comprises administering to a subject an effective amount of a compound according to Formula I, or a pharmaceutically acceptable salt thereof.

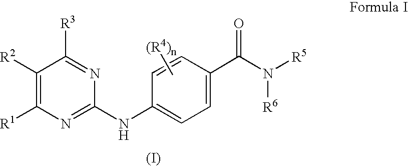

Compounds of this type have been shown to significantly impair NASH progression in an animal model of dietary induced NASH. Specifically, the administration of compounds according to Formula I to mice with dietary induced NASH resulted in a clear improvement in obesity (Example 1), insulin resistance (Example 2), visceral fat accumulation (Example 3), inflammation (Example 4), lipid accumulation (Example 5), lipid catabolism (Example 6), oxidative stress (Example 7) and hepatocyte apoptosis and liver fibrosis (Example 8).

A preferred embodiment of the present invention is a method of treating a subject with nonalcoholic fatty liver disease (NAFLD)/non-alcoholic steatohepatitis (NASH) comprising administering to the subject an effective amount of compound A:

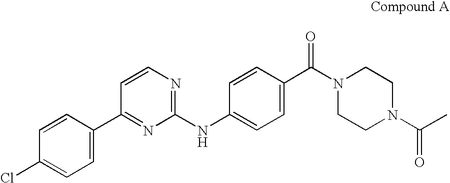

or a physiologically acceptable salt thereof.

In another embodiment, the present invention is a method of treating insulin resistance in a subject, comprising administering to the subject an effective amount of a compound according to Formula I, or a physiologically acceptable salt thereof, wherein the subject is suffering from a disorder other than type II diabetes.

In another embodiment, the present invention is a method of treating obesity in a subject, comprising administering to the subject an effective amount of a compound according to Formula I, or a physiologically acceptable salt thereof.

In another embodiment the present invention is a method of treating hyperlipidemia in a subject, comprising administering to the subject an effective amount of a compound according to Formula I, or a physiologically acceptable salt thereof.

In another embodiment the present invention is a method of treating alcoholic steatohepatitis in a subject, comprising administering to the subject an effective amount of a compound according to Formula I, or a physiologically acceptable salt thereof.

In another embodiment the present invention is a method of treating acute liver failure in a subject, comprising administering to the subject an effective amount of a compound according to Formula I, or a physiologically acceptable salt thereof.

### BRIEF DESCRIPTION OF THE DRAWINGS

FIG. 1, shows bar graphs comparing the percent weight gain, serum glucose and serum insulin in in a) control mice feed with the HSD diet b) mice feed with the HSD diet and treated with compound 1 and c) mice feed a chow diet and treated with vehicle only. The graphs show the effects of activity of compound A on (A) body weight gain, the rise in (B) blood glucose and the rise in (C) serum insulin levels. Values are a mean of at less 4 animals per time point and treatment. *p<0.05 (vehicle vs control), #p<0.05 (vehicle vs Compound 1).

FIG. 2, is a bar graph of the effect of compound A on HSD induced hepatic steatosis, comparing the steatosis score in a) control mice feed with the HSD diet b) mice feed with the HSD diet and treated with compound A and c) mice feed a chow diet and treated with vehicle only. Values are a mean of at least 4 animals per time point and treatment group. **p<0.01 (vehicle vs control), ##p<0.01 (vehicle vs Compound A).

FIG. 3. shows bar graphs of the effects of compound A in HSD induced NF-κB activation and proinflammatory cytokine expression. (A) and (B) show IL-6 and TNF expression analysed by Real Time RT-PCR in a) control mice feed with the HSD diet b) mice feed with the HSD diet and treated with compound A and c) mice feed a chow diet and treated with vehicle only. (C) and (D) show serum and liver adiponectin expression by ELISA in a) control mice feed with the HSD diet b) mice feed with the HSD diet and treated with compound A and c) mice feed a chow diet and treated with vehicle only. All Real time values are standardized with GAPDH values and expressed in times vs control. Values are a mean of at least 4 animals per time point and treatment group. *p>0.05; **p<0.01 (vehicle vs control), #p<0.05; ##p<0.01 (vehicle vs Compound A).

FIG. 4. shows bar graphs of the effects of HSD/compound A on lipid catabolism related genes in a) control mice feed with the HSD diet b) mice feed with the HSD diet and treated with compound A and c) mice feed a chow diet and treated with vehicle only (A) shows mRNA levels measured by real time RT-PCR for PPARα expression; (B) and (C) show CPT-1 and Acyl-CoA oxidase mRNA expression; (D) shows PPARγ on the mRNA and protein level; (E) shows fatty acid translocase/CD36 gene expression. All Real time values are internally corrected with GAPDH values and expressed in times vs control. Values are a mean of at least 4 animals per time point and treatment group. *p<0.05; **p<0.01 (compound A vs control), #p<0.05; ##p<0.01 (Compound A vs vehicle).

FIG. 5. shows bar graphs of the effects of compound A on HSD triggered mitochondrial dysfunction and oxidative stress in a) control mice feed with the HSD diet b) mice feed with the HSD diet and treated with compound A and c) mice feed a chow diet and treated with vehicle only. (A) shows Real time RT-PCR of CYP2E1 upregulation; (B) shows real time RT-PCR of MnSOD and (C) shows catalase mRNA levels. *p<0.05 (compound A vs control); #p<0.05, ##P<0.01 (vehicle vs compound A), +p<0.05 (vehicle vs control).

FIG. 6. shows bar graphs of the effects of compound A on the progression of NASH by blocking HSD induced hepatocyte apoptosis and liver fibrosis in a) control mice feed with the HSD diet b) mice feed with the HSD diet and treated with compound A and c) mice feed a chow diet and treated with vehicle only. (A) shows caspase-3 activity measured in whole liver extracts and (B) shows fibrosis assessed in histological sections. **p<0.01 (vehicle vs control), #p<0.05, ##p<0.01 (vehicle vs compound A).

### DETAILED DESCRIPTION OF THE INVENTION

“Aryl” refers to an unsaturated aromatic carbocyclic group of from 6 to 14 carbon atoms having a single ring (e.g. phenyl) or multiple condensed rings (e.g. naphthyl). Preferred aryls include phenyl, naphthyl, phenantrenyl and the like.

“Alkylaryl” refers to an alkyl having at least one alkyl hydrogen atom replaced with an aryl moiety, such as benzyl, —(CH2)2phenyl, —(CH2)3phenyl, —CH(phenyl)2, and the like.

“Alkyl” refers to a straight chain or branched, saturated or unsaturated alkyl, cyclic or non-cyclic hydrocarbon having from 1 to 10 carbon atoms, while “lower alkyl” or “C₁-C₆-alkyl” has the same meaning, but only has from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Unsaturated alkyls contain at least one double or triple bond between adjacent carbon atoms (also referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl, 2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like. Representative saturated “cyclic alkyls” include cyclopropyl, cyclobutyl, cyclopentyl, cyclohexyl, and the like; while unsaturated cyclic alkyls include cyclopentenyl and cyclohexenyl, and the like. Cycloalkyls are also referred to herein as “carbocyclic” rings systems, and include bi- and tri-cyclic ring systems having from 8 to 14 carbon atoms, such as a cycloalkyl (such as cyclopentane or cyclohexane) fused to one or more aromatic (such as phenyl) or non-aromatic (such as cyclohexane) carbocyclic rings.

“Alkoxy” refers to -O-(alkyl) or -O-aryl), such as methoxy, ethoxy, n-propyloxy, iso propyloxy, n-butyloxy, iso-butyloxy, phenoxy and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (—CH₂CH═CH₂) and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C═CH), propargyl (—CH₂C═CH), and the like.

“Halogen” refers to fluorine, chlorine, bromine or iodine.

“Keto” refers to a carbonyl group (i. e.,C═O).

“Heteroaryl” refers to an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, and quinazolinyl.

“Heteroalkylaryl” refers to an alkyl having at least one alkyl hydrogen atom replaced with a heteroaryl moiety, such as —CH₂-pyridinyl, —CH₂-pyrimidinyl, and the like.

“Heterocycloalkyl” or “heterocycle” refers to a heterocyclic ring containing from 5 to 10 ring atoms. Specifically, a 5- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Alkylheterocycloalkyl” refers to an alkyl having at least one alkyl hydrogen atom replaced with a heterocycle, such as 2-(1-pyrrolidinyl)ethyl, 4-morpholinylmethyl, (1-methyl-4-piperidinyl)methyl and the like.

The term “substituted” as used herein refers to any of the above groups (i. e. alkyl, aryl, alkyl aryl, heterocyclyl and heterocycloalkyl) wherein at least one hydrogen atom is replaced with a substituent. In the case of a keto substituent (“C(═O)”) two hydrogen atoms are replaced. Substituents include halogen, hydroxy, alkyl, substituted alkyl (such as haloalkyl, mono- or all-substituted aminoalkyl, alkyloxyalkyl, and the like), aryl, substituted aryl, arylalkyl, substituted arylalkyl, heterocycloalkyl, substituted heterocycloalkyl, alkylheterocycloalkyl, substituted alkylheterocycloalkyl, —NRaRb, —NRₐC(═O)R_{b}, —NRₐC(═O)NRₐR_{b}, NRₐC(═O)OR_{b} —NRₐSO₂R_{b}, —ORₐ, —C(═O)Rₐ, —C(═O)ORₐ, —C(═O)NRₐR_{b}, —OC(═O)Rₐ, —OC(═O)ORₐ, —OC(═O)NRₐR_{b}, —NRₐSO₂R_{b}, or a radical of the formula Y-Z-Rₐ where Y is alkanediyl, substituted alkanediyl, or a direct bond, Z is —O—, —S—, S(═O)—, —S(═O)₂—, —N(R_{b})—, —C(═O)—, —C(═O)O—, —OC(═O)—, —N(R_{b})C(═O)—, —C(═O)N(R_{b})— or a direct bond, wherein Rₐ and R_{b} are the same or different and independently hydrogen, amino, alkyl, substituted alkyl (including halogenated alkyl), aryl, substituted aryl, alkylaryl, substituted alkylaryl, heterocycloalkyl, substituted heterocycloalkyl, alkylheterocyloalkyl or substituted alkylheterocycloalkyl, or wherein Rₐ and R_{b} taken together with the nitrogen atom to which they are attached form a heterocycle or substituted heterocycle.

“Haloalkyl” refers to an alkyl having one or more hydrogen atoms replaced with halogen, such as —CF₃.

“Hydroxyalkyl” means alkyl having one or more hydrogen atoms replaced with hydroxy, such as —CH₂OH.

“Sulfonyl” refers to group “—SO₂-R” wherein R is selected from H, aryl, heteroaryl, C₁-C₆-alkyl, C₁-C₆-alkyl substituted with halogens,( e.g., an —S0₂—CF₃ group), C₂-C₆-alkenyl, C₂-C₆-alkynyl, C₃-C₈-cycloalkyl, heterocycloalkyl, aryl, heteroaryl, C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynylheteroaryl, C₁-C₆-alkyl cycloalkyl, or C₁-C₆-alkyl heterocycloalkyl.

“Sulfinyl” refers to a group “—S(O)—R” wherein R is selected from H, C₁-C₆-alkyl, C₁-C₆-alkyl substituted with halogens,( e.g., an —SO₂—CF₃ group), C₂-C₆-alkenyl, C₂-C₆-alkynyl, C₃-C₈-cycloalkyl, heterocycloalkyl, aryl, heteroaryl, C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynylheteroaryl, C₁-C₆-alkyl cycloalkyl, or C₁-C₆-alkyl heterocycloalkyl.

“Sulfanyl” refers to groups “—S—R” where R is selected from H, C_-C₆-alkyl, C₁-C₆-alkyl substituted with halogens,( e.g., an —SO₂—CF₃ group), C₂-C₆-alkenyl, C₂-C₆-alkynyl, C₃-C₈-cycloalkyl, heterocycloalkyl, aryl, heteroaryl, C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynylheteroaryl, C₁-C₆-alkyl cycloalkyl, or C₁-C₆-alkyl heterocycloalkyl. Preferred sulfanyl groups include methylsulfanyl, ethylsulfanyl, and the like.

“Carboxyl” refers —COOH.

“Amino” refers to the group —NRR′ where each R, R′ is independently hydrogen or C₁-C₆-alkyl, aryl, heteroaryl, C₁-C₆-alkyl aryl, C₁-C₆-alkyl heteroaryl, cycloalkyl, or heterocycloalkyl, and where R and R′, together with the nitrogen atom to which they are attached, can optionally form a 3-8-membered heterocycloalkyl ring.

“Ammonium” refers to a positively charged group −N⁺RR′R″, where each R, R′, R″ is independently C₁-C₆-alkyl, C₁-C₆-alkyl aryl, C₁-C₆-alkyl heteroaryl, cycloalkyl, or heterocycloalkyl, and where R and R′, together with the nitrogen atom to which they are attached, can optionally form a 3-8-membered heterocycloalkyl ring.

“HCl” means the hydrochloride salt of compounds depicted by their chemical structure.

“Nitrogen-containing non-aromatic heterocycle” means morpholinyl, thiomorpholinyl, pyrrolidinonyl, pyrrolidinyl, piperidinyl, homopiperidinyl, piperazinyl, homopiperazinyl, hydantoinyl, tetrahydropyrindinyl, tetrahydropyrimidinyl, oxazolidinyl, thiazolidinyl, indolinyl, isoindolinyl, tetrahydroquinolinyl, tetrahydroisoquinolinyl and the like.

“Pharmaceutically acceptable salts or complexes” refer to salts or complexes of the compounds disclosed herein. Examples of such salts include, but are not limited to, salts which are formed with inorganic acids (e.g. hydrochloric acid, hydrobromic acid, sulfuric acid, phosphoric acid, nitric acid, and the like), as well as salts formed with organic acids such as acetic acid, oxalic acid, tartaric acid, succinic acid, malic acid, fumaric acid, maleic acid, ascorbic acid, benzoic acid, tannic acid, palmoic acid, alginic acid, polyglutamic acid, naphthalene sulfonic acid, methane sulfonic acid, naphthalene disulfonic acid, and poly-galacturonic acid, as well as salts formed with basic amino acids such as lysine or arginine.

Additionally, salts of compounds containing a carboxylic acid or other acidic functional group(s) can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, N-benzyl-β-phenethylamine, dehydroabietylamine, N,N′-bisdehydroabietylamine, glucamine, N-methylglucamine, collidine, quinine, quinoline, and basic amino acid such as lysine and arginine.

The present invention is directed to a method of treating a subject with nonalcoholic fatty liver disease (NAFLD) including fatty liver (steatosis), nonalcoholic steatohepatitis (NASH), and cirrhosis (advanced scarring of the liver). The method comprises administering to a subject an effective amount of a compound according to Formula I, or a pharmaceutically acceptable salt thereof.

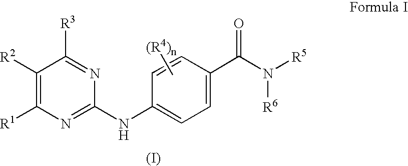

One function of the liver is to process fats and proteins from digested food. Fatty liver disease covers a range of conditions where there is a build-up of fat in the liver cells. The liver cells (hepatocytes) normally contain some fat and related fatty chemicals (triglycerides, fatty acids, etc). Excess fat is normally passed out of liver cells, into the bloodstream, and then taken up and stored in fat cells (adipose cells) throughout the body. In fatty liver disease, excess fat builds up in liver cells. This is thought to happen if there is some problem or disruption in the normal processing of fat and related fatty chemicals in the liver cells. Simple fatty liver (also called “hepatic steatosis”) is present when the fat content inside liver cells makes up more than 5-10% of the liver's weight. Simple fatty liver is not associated with serious damage or harm to the liver.

Nonalcoholic fatty liver disease (NAFLD) refers to a wide spectrum of liver disease ranging from:
i) simple fatty liver (steatosis), in which there are fat deposits on the liver;
ii) nonalcoholic steatohepatitis (NASH) in which there are fat deposits on the liver along with inflammation and damage of the liver; and
iii) cirrhosis in which there is irreversible, advanced scarring of the liver.

All of the stages of NAFLD have in common the accumulation of fat (fatty infiltration) in the liver cells (hepatocytes). Fatty liver (steatosis) can progress to nonalcoholic steatohepatitis (NASH). In NASH, the fat accumulation is associated with varying degrees of inflammation and scarring of the liver, and in many cases insulin resistance, dyslipidemia and hypertension. NASH can progress to fibrosing, steatohepatitis and trigger cirrhosis, end-stage liver disease, acute live failure and hepatocellular carcinoma. It most often occurs in people with excess body weight, elevated blood lipids, such as cholesterol and triglycerides, and insulin resistance.

The present invention also provides methods of treating acute liver failure. Acute liver failure occurs when the cells in the liver die or become damaged in a short period of time. This causes the liver to fail to work normally and can be fatal. Any progressive liver disease, such as cirrhosis, can result in liver failure. Signs of liver failure include encephalopathy (altered brain function, jaundice, ascites fetor hepaticus and failure of coagulation).

Many people with simple fatty liver have other conditions where fatty liver is a complication. Many cases of simple fatty liver develop in people who drink more alcohol than the recommended limits. Over half of people who drink heavily develop simple fatty liver. In these cases simple fatty liver can progress to alcoholic steatohepatitis. In this condition the excess fat in the liver cells is associated with, or may cause, inflammation of the liver. Alcoholic steatohepatitis, may eventually cause scarring (cirrhosis) of the liver.

Insulin resistance is an impaired metabolic response to our body's own insulin, so that active muscle cells cannot take up glucose as easily as they should. As a result, glucose is prevented from entering the cells and remains in the blood stream. The pancreas tries to keep up with the demand by producing more insulin. Eventually, the pancreas cannot keep up with the body's need for insulin, and excess glucose builds up in the bloodstream. Many people with insulin resistance have high levels of blood glucose and high levels of insulin circulating in their blood at the same time.

People with blood glucose levels that are higher than normal but not yet in the diabetic range have “pre-diabetes” (impaired fasting glucose (IFG) or impaired glucose tolerance (IGT)). Pre-diabetes increases the risk of developing type 2 diabetes.

Prediabetes can be detected by either of the two standard tests currently used to diagnose diabetes. In the fasting plasma glucose test (FPG), a normal fasting blood glucose level is under about 100 mg/dl, and fasting blood glucose in the range of about 100 to about 125 mg/dl indicates impaired fasting glucose (IFG), or prediabetes. A fasting blood glucose level over about 125 mg/dl indicates diabetes. In the oral glucose tolerance test (OGTT), a normal blood glucose would be below about 140 mg/dl; an elevated blood glucose level in the range of about 140 to about 199 mg/dl indicates impaired glucose tolerance, or prediabetes. A blood glucose level of about 200 mg/dl or higher indicates diabetes.

Gestational diabetes is a condition in which the glucose level is elevated and other diabetic symptoms appear during pregnancy in a woman who has not previously been diagnosed with diabetes. Diabetic symptoms typically disappear following delivery. Gestational diabetes is caused by blocking effects of other hormones on the insulin that is produced by the pancreas (insulin resistance).

Insulin resistance is also associated with syndrome X. Syndrome X is a combination of metabolic disorders. Specifically, syndrome X, which is also known as the “metabolic syndrome”, “Insulin Resistance Syndrome”, or “metabolic syndrome X”, refers to a groups of risk factors for heart disease that seem to cluster in some people. It is defined as the presence of three or more of the following conditions:
i) insulin resistance or glucose intolerance
ii) elevated blood pressure
iii) elevated triglycerides
iv) low levels of HDL (high density cholesterol) cholesterol
v) central (abdominal) obesity

Other symptoms of Syndrome X may include prothrombotic state (e.g., high fibrinogen or plasminogen activator inhibitor in the blood) and proinflammatory state (e.g., elevated high-sensitivity C-reactive protein in the blood).

For adults, overweight and obesity ranges are determined by using weight and height to calculate a number called the “body mass index” (BMI). Body Mass Index (BMI) is a number calculated from a person's weight and height, using the formula: weight (kg)/[height (m)]² (calculation: [weight (kg)/height (m)/height (m)]). With the metric system, the formula for BMI is weight in kilograms divided by height in meters squared. BMI is used because, for most people, it correlates with their amount of body fat. An adult who has a BMI between 25 and 29.9 is considered overweight. An adult who has a BMI of 30 or higher is considered obese.

Hyperlipidemia is an elevation of lipids (fats) in the bloodstream. These lipids include cholesterol, cholesterol esters (compounds), phospholipids and triglycerides. They're transported in the blood as part of large molecules called lipoproteins. When hyperlipidemia is defined in terms of a class or classes of elevated lipoproteins in the blood, the term hyperlipoproteinemia is used. Hypercholesterolemia is the term for high cholesterol levels in the blood. Hypertriglyceridemia refers to high triglyceride levels in the blood.

The present invention provides methods for treating all of the above listed diseases, disorders and syndromes using a compound according to Formula I. Effective amounts of such compounds are administered to a subject with one or more of these conditions.

As used herein “treating” includes achieving, partially or substantially, one or more of the following results: partially or totally reducing the extent of the disease, disorder or syndrome (e.g., reducing fat deposits, increasing insulin activity, reducing weight); ameliorating or improving a clinical symptom or indicator associated with the disorder; delaying, inhibiting or preventing the progression of the disease, disorder or syndrome; or partially or totally delaying, inhibiting or preventing the onset or development of disorder. Delaying, inhibiting or preventing the progression of the disease, disorder or syndrome includes for example, delaying, inhibiting or preventing the progression of fatty liver to NASH; delaying, inhibiting or preventing the progression of NASH to cirrhosis, end-stage liver disease and/or hepatocellular carcinoma; and delaying, inhibiting or preventing the progression of pre-diabetes to diabetes.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

Compounds according to Formula I can be used alone or in combination e.g., as an adjunct therapy, with at least one other therapeutic agent. Compounds according to Formula I can be administered concurrently with the administration of the other therapeutic drug, which can be part of the same composition as or in a different composition from that comprising the claimed compound. Alternatively, compounds according to Formula I can be administered prior to or subsequent to administration of another therapeutic agent

When used in the methods of the present invention to treat a subject with NASH, a compound according to Formula I can be co-administered with a therapeutic agent used to reduce one or more of the symptoms of NASH including, but not limited to, an agent used to control blood glucose levels, an agent used to control lipid levels, e.g., an agent used to lower control cholesterol, an antioxidant, an appetite suppressing agent, an anti-obesity agent an antibiotic or an anti-inflammatory agent. Examples of such agents are listed herein and also include an agent used to control blood glucose levels, such as, sulfonylureas, such as, chlorpropamide (brand name Diabinese), glipizide (brand names Glucotrol and Glucotrol XL), glyburide (brand name Micronase, Glynase, and Diabeta), and glimepiride (Amaryl); meglitinides, such as, repaglinide (brand name Prandin) and nateglinide (brand name Starlix); biguanides, such as, metformin (brand name Glucophage®) and acarbose (Precose); thiazolidinediones, such as, rosiglitazone (brand name Avandia®), troglitazone (brand name Rezulin®), and pioglitazone (brand name Actos®); alpha-glucosidase inhibitors, such as, acarbose (brand name Precose®) and meglitol (brand name Glyset); and insulin, such as, pramlintide (brand name Symlin), exenatide (brand name Byetta), humalog (brand name Lispro), novolog (brand name Aspart), humulin, novolin, ultralente, and lantus (brand name Glargine); an agent used to control lipid levels, such as, vytorin, LXR agonists (see WO 01/03705 the entire content of which are incorporated herein by reference), Clofibrate and Gemfibrozil, a plasma HDL-raising agent, a cholesterol lowering agent, such as, ursodeoxycholic acid (a synthetic bile salt Actigall®, URSO®, or Ursodiol®), a cholesterol biosynthesis inhibitor, for example an HMG-CoA reductase inhibitor (such as a statin, such as, Atorvastatin (Lipitor) Fluvastatin (Lescol) Lovastatin (Altocor, Mevacor) Pravastatin (Pravachol) Rosuvastatin (Crestor) Simvastatin (Zocor) and rosuvastatin calcium), an HMG-CoA synthase inhibitor, a squalene epoxidase inhibitor, or a squalene synthetase inhibitor (also known as squalene synthase inhibitor), an acyl-coenzyme A: cholesterol acyltransferase (ACAT) inhibitor, such as, melinamide; probucol, niacin (nicotinic acid, Vitamin-B-3), nicotinic acid and the salts thereof and niacinamide; a cholesterol absorption inhibitor such as beta-sitosterol, and exetimibe (Zatia), a bile acid sequestrant, such as, cholestyramine(Questran), colestipol (Colestid), and Colesevelam (WelChol), or a dialkylaminoalkyl derivatives of a cross-linked dextran; and LDL (low density lipoprotein) receptor inducer, fibrates such as clofibrate, fenofibrate, and gemfibrizol, vitamin B₆ (also known as pyridoxine) and physiologically acceptable salts thereof, such as the HCl salt; vitamin B₁₂ (also known as cyanocobalamin), and angiotensin II antagonist converting enzyme inhibitor; and a platelet aggregation inhibitor, such as fibrinogen receptor antagonists (i.e., glycoprotein IIb/IIIa fibrinogen receptor antagonists); an antibiotic, such as, Polymixin B; and an antioxidant, such as, selenium, betaine, vitamin C, vitamin E and beta carotene: a beta-blocker; an agent used to reduce weight or suppress appetite, such as, sibutramine (Meridia), orlistat, (Xenical), anorectics (Anorexigenics), dexedrine, digoxin, cannabinoid (CB 1) receptor antagonists, rimonabant, amphetamines, lipase inhibitors, bupropion, topiramate, zonisamide, fenfluramine, phentermine (Adipex-P, Fastin, lonamin, Oby-trim, Pro-Fast, Zantryl), phendimetrazine (Bontril, Plegine, Prelu-2, X-Trozine, Adipost), diethylpropion (Tenuate, Tenuate dospan), fluoxetine/phentermine, phendimetrazine/phentermine, and orlistat/sibutramine.

When used in the methods of the present invention to treat a subject with alcoholic steatohepatitis, a compound according to Formula I can be co-administered with a therapeutic agent used to reduce one or more of the symptoms of alcoholic steatohepatitis including, but not limited to, an agent used to control blood glucose levels, an agent used to control lipid levels, e.g., an agent used to lower control cholesterol, an antioxidant, an appetite suppressing agent, an anti-obesity agent an antibiotic or an anti-inflammatory agent, such as those described above.

When used in the methods of the present invention to treat a subject with acute liver failure, a compound according to Formula I can be co-administered with a therapeutic agent used to reduce one or more of the symptoms of alcoholic steatohepatitis including, but not limited to, an agent used to control blood glucose levels, an agent used to control lipid levels, e.g., an agent used to lower control cholesterol, an antioxidant, an appetite suppressing agent, an anti-obesity agent an antibiotic or an anti-inflammatory agent, such as those described above.

When used in the methods of the present invention to treat a subject with insulin resistance, a compound according to Formula I can be co-administered with a therapeutic agent used to control blood glucose levels. Examples of such compounds include, sulfonylureas, such as, chlorpropamide (brand name Diabinese), glipizide (brand names Glucotrol and Glucotrol XL), glyburide (brand name Micronase, Glynase, and Diabeta), and glimepiride (Amaryl); meglitinides, such as, repaglinide (brand name Prandin) and nateglinide (brand name Starlix); biguanides, such as, metformin (brand name Glucophage®) and acarbose (Precose); thiazolidinediones, such as, rosiglitazone (brand name Avandia®), troglitazone (brand name Rezulin®), and pioglitazone (brand name Actos®); alpha-glucosidase inhibitors, such as, acarbose (brand name Precose®) and meglitol (brand name Glyset); and insulin, such as, pramlintide (brand name Symlin), exenatide (brand name Byetta), humalog (brand name Lispro), novolog (brand name Aspart), humulin, novolin, ultralente, and lantus (brand name Glargine).

When used in the methods of the present invention to treat a subject with gestational diabetes, a compound according to Formula I can be co-administered with a therapeutic agent used to control blood glucose levels. Examples includes those listed in the previous paragraph.

When used in the methods of the present invention to treat a subject with syndrome X, a compound according to Formula I can be co-administered with a therapeutic agent used to treat one or more of the symptoms of syndrome X, including but not limited to, an agent used to control blood glucose levels, an agent used to control lipid levels, e.g., an agent used to lower control cholesterol, an appetite suppressing agent, an anti-obesity agent, an antioxidant, an antibiotic or an anti-inflammatory agent. Examples of such agents are listed herein and also include an agent used to control blood glucose levels, such as, sulfonylureas, such as, chlorpropamide (brand name Diabinese), glipizide (brand names Glucotrol and Glucotrol XL), glyburide (brand name Micronase, Glynase, and Diabeta), and glimepiride (Amaryl); meglitinides, such as, repaglinide (brand name Prandin) and nateglinide (brand name Starlix); biguanides, such as, metformin (brand name Glucophage®) and acarbose (Precose); thiazolidinediones, such as, rosiglitazone (brand name Avandia®), troglitazone (brand name Rezulin®), and pioglitazone (brand name Actos®); alpha-glucosidase inhibitors, such as, acarbose (brand name Precose®) and meglitol (brand name Glyset); and insulin, such as, pramlintide (brand name Symlin), exenatide (brand name Byetta), humalog (brand name Lispro), novolog (brand name Aspart), humulin, novolin, ultralente, and lantus (brand name Glargine); an agent used to control lipid levels, such as, vytorin, LXR agonists (see WO 01/03705 the entire content of which are incorporated herein by reference), Clofibrate and Gemfibrozil, a plasma HDL-raising agent, a cholesterol lowering agent, such as, ursodeoxycholic acid (a synthetic bile salt Actigall®, URSO®, or Ursodiol®), a cholesterol biosynthesis inhibitor, for example an HMG-CoA reductase inhibitor (such as a statin, such as, Atorvastatin (Lipitor) Fluvastatin (Lescol) Lovastatin (Altocor, Mevacor) Pravastatin (Pravachol) Rosuvastatin (Crestor) Simvastatin (Zocor) and rosuvastatin calcium), an HMG-CoA synthase inhibitor, a squalene epoxidase inhibitor, or a squalene synthetase inhibitor (also known as squalene synthase inhibitor), an acyl-coenzyme A: cholesterol acyltransferase (ACAT) inhibitor, such as, melinamide; probucol, niacin (nicotinic acid, Vitamin-B-3), nicotinic acid and the salts thereof and niacinamide; a cholesterol absorption inhibitor such as beta-sitosterol, and exetimibe (Zatia), a bile acid sequestrant, such as, cholestyramine(Questran), colestipol (Colestid), and Colesevelam (WelChol), or a dialkylaminoalkyl derivatives of a cross-linked dextran; and LDL (low density lipoprotein) receptor inducer, fibrates such as clofibrate, fenofibrate, and gemfibrizol, vitamin B₆ (also known as pyridoxine) and physiologically acceptable salts thereof, such as the HCl salt; vitamin B₁₂ (also known as cyanocobalamin), and angiotensin II antagonist converting enzyme inhibitor; and a platelet aggregation inhibitor, such as fibrinogen receptor antagonists (i.e., glycoprotein IIb/IIIa fibrinogen receptor antagonists); and an antioxidant, such as, selenium, betaine, vitamin C, vitamin E and beta carotene: a beta-blocker; an agent used to reduce weight or suppress appetite, such as, sibutramine (Meridia), orlistat, (Xenical), anorectics (Anorexigenics), dexedrine, digoxin, cannabinoid (CBl) receptor antagonists, rimonabant, amphetamines, lipase inhibitors, bupropion, topiramate, zonisamide, fenfluramine, phentermine (Adipex-P, Fastin, lonamin, Oby-trim, Pro-Fast, Zantryl), phendimetrazine (Bontril, Plegine, Prelu-2, X-Trozine, Adipost), diethylpropion (Tenuate, Tenuate dospan), fluoxetine/phentermine, phendimetrazine/phentermine, and orlistat/sibutramine; and aspirin.

When used in the methods of the present invention to treat a subject with hyperlipidemia, a compound according to Formula I can be co-administered with a therapeutic agent used to control lipid levels, such as those described above.

When used in the methods of the present invention to treat a subject with obesity, a compound according to Formula I can be administered with a therapeutic agent used to reduce weight or suppress appetite. Examples include those selected from the group consisting of sibutramine (Meridia), orlistat, (Xenical), anorectics (Anorexigenics), dexedrine, digoxin, cannabinoid (CBl) receptor antagonists, rimonabant, amphetamines, lipase inhibitors, bupropion, topiramate, zonisamide, fenfluramine, phentermine (Adipex-P, Fastin, lonamin, Oby-trim, Pro-Fast, Zantryl), phendimetrazine (Bontril, Plegine, Prelu-2, X-Trozine, Adipost), diethylpropion (Tenuate, Tenuate dospan), fluoxetine/phentermine, phendimetrazine/phentermine, and orlistat/sibutramine.

The method of treatment of a subject suffering from non-alcoholic fatty liver disease (NAFLD)/non-alcoholic steatohepatitis (NASH) comprises administration to the subject of an effective amount of a compound which is an anilinopyrimidine derivative of Formula (I)

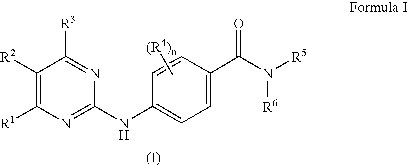

Said compounds are disclosed in WO 02/46171 (Signal Pharmaceuticals Inc.), which are described in particular for the treatment of autoimmune disorders, inflammatory diseases, cardiovascular diseases, infectious diseases, stroke or cancer.

In said compounds according to Formula (I), which include its pharmaceutically acceptable salts thereof, the substituents are defined as follows:

R¹ is either an aryl or heteroaryl optionally substituted with one to four substituents independently selected from R⁷;

R² is hydrogen;

R³ is either hydrogen or lower alkyl;

R⁴ is, in each instance, independently selected from the group consisting of halogen, hydroxy, lower alkyl and lower alkoxy; and wherein n is an integer from 0-4;

R⁵ and R⁶ are the same or different and are independently selected from the group consisting of —R⁸, —(CH₂)ₐC(═O)R⁹) —(CH₂)ₐC(═O)OR⁹—(CH₂)ₐC(═O)NR⁹R¹⁰, —(CH₂)ₐC(═O)NR⁹(CH₂)_{b}C(═O)R¹⁰, —(CH₂)ₐNR⁹C(═O)R¹⁰, —(CH₂)ₐNR¹¹C(═O)NR⁹R¹⁰, —(CH₂)ₐNR⁹R¹⁰, —(CH₂)ₐOR⁹, —(CH₂)ₐSO_{c}R⁹ or —(CH₂)ₐSO₂NR⁹R¹⁰; and R⁵ and R⁶ taken together with the nitrogen atom to which they are attached to form a heterocycle or substituted heterocycle;

R⁷ is at each occurrence independently selected from the group consisting of halogen, hydroxy, cyano, nitro, carboxy, alkyl, alkoxy, haloalkyl, acyloxy, sulfanylalkyl, sulfinylalkyl, sulfonylalkyl, hydroxyalkyl, aryl, substituted aryl, alkylaryl, substituted alkylaryl, heterocycloalkyl, substituted heterocycloalkyl, alkylheterocycloalkyl, substituted alkylheterocycloalkyl, —C(═O)OR⁸, —OC(═O)R⁸, —C(═O)NR⁸R⁹, —C(═O)NR⁸OR⁹, -SO_{C}R⁸, —SO_{C}NR⁸R⁹, —NR⁸SO_{C}R⁹, —NR⁸R⁹, —NR⁸C(═O)R⁹, —NR⁸C(═O)(CH₂)_{b}OR⁹, —NR⁸C(═O)(CH₂)_{b}R⁹, —O(CH₂)_{b}NR⁸R⁹ and substituted or unsubstituted heterocycloalkyl fused to substituted or unsubstituted phenyl;

R⁸, R⁹, R¹⁰ and R¹¹ are the same or different and are at each occurrence independently selected from the group consisting of hydrogen, alkyl, substituted alkyl, aryl, substituted aryl, alkylaryl, substituted alkylaryl, heterocycloalkyl, substituted heterocycloalkyl, alkylheterocycloalkyl and substituted alkylheterocycloalkyl;
or R⁸ and R⁹ taken together with the atom or atoms to which they are attached to form a heterocycle or substituted heterocycle;
a and b are the same or different and are at each occurrence independently selected from the group consisting of 0, 1, 2, 3 or 4; and
c is at each occurrence 0, 1 or 2.

In one embodiment of the invention, R¹ is either a substituted or unsubstituted aryl or heteroaryl. When R¹ is substituted, it is substituted with one or more substituents defined below. Preferably, when substituted, R¹ is substituted with a halogen, sulfonyl or sulfonamide.

In another embodiment of the invention, R¹ is selected from the group consisting of a substituted or unsubstituted aryl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl and quinazolinyl.

In another embodiment of the invention, R¹ is a substituted or unsubstituted aryl, preferably a substituted or unsubstituted phenyl. When R¹ is a substituted aryl, the aryl is substituted with one or more substituents defined below.

Preferably, when R¹ is a substituted aryl, it is substituted with a halogen, sulfonyl or sulfonamide.

In another embodiment of the invention, R⁵ and R⁶, taken together with the nitrogen atom to which they are attached form a substituted or unsubstituted nitrogen-containing non-aromatic heterocycle, preferably substituted or unsubstituted morpholinyl, substituted or unsubstituted thiomorpholinyl, substituted or unsubstituted pyrrolidinonyl, substituted or unsubstituted pyrrolidinyl, substituted or unsubstituted piperidinyl, substituted or unsubstituted homopiperidinyl, substituted or unsubstituted piperazinyl, substituted or unsubstituted homopiperazinyl, substituted or unsubstituted hydantoinyl, substituted or unsubstituted tetrahydropyrindinyl, substituted or unsubstituted tetrahydropyrimidinyl, substituted or unsubstituted oxazolidinyl, substituted or unsubstituted thiazolidinyl, substituted or unsubstituted indolinyl, substituted or unsubstituted isoindolinyl, substituted or unsubstituted tetrahydroquinolinyl or substituted or unsubstituted tetrahydroisoquinolinyl.

When R5 and R6, taken together with the nitrogen atom to which they are attached form a substituted or unsubstituted piperazinyl, a substituted or unsubstituted piperadinyl or a substituted or unsubstituted morpholinyl, the substituted piperazinyl, substituted piperadinyl or substituted morpholinyl is substituted with one or more substituents defined below.

Preferably, when substituted, the substituent is alkyl, amino, alkylamino, alkylether, acyl, pyrrolidinyl or piperidinyl.

In one embodiment of the invention, R², R³ and R⁴ are hydrogen, and the compounds of this invention has the following Formula (II):

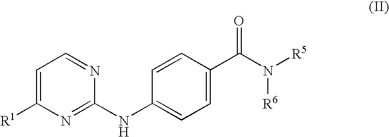

In a more specific embodiment of the invention, R¹ is a phenyl optionally substituted with R⁷, and having the following Formula (III):

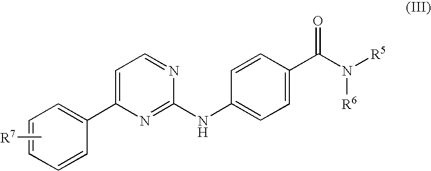

In still a further embodiment of the invention, R⁷ is at the para position of the phenyl ring, as represented by the following Formula (IV):

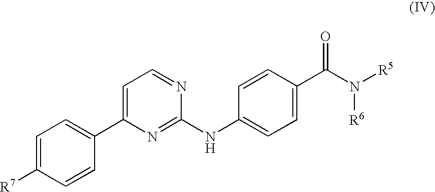

In still a further embodiment of the invention, in the anilinopyrimidine derivatives is compound A: 1-(4-{4-[4-(4-Chloro-phenyl)-pyrimidin-2-ylamino]-benzoyl}-piperazin-1-yl)-ethanone.

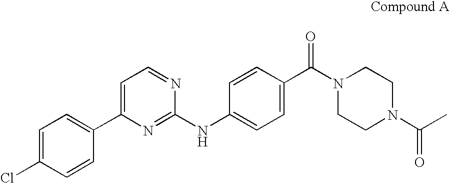

Compounds according to Formulae I-IV and compound A can be prepared by methods described in WO 02/46171 A2, (Signal Pharmaceuticals Inc.) the entire contents of which are incorporated herein by reference.

Furthermore, the invention provides pharmaceutical compositions comprising compounds according to Formulae I, II, III, IV, or compound A, or a pharmaceutically acceptable salt thereof, as active ingredient together with a pharmaceutically acceptable carrier.

Another embodiment of the invention is a compound of formula (I), (II), (III) or (V) or a pharmaceutically acceptable salt thereof, provided that compound A or a pharmaceutically acceptable salt thereof is excluded.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, a compound according to Formula I can be combined as the active ingredient in admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compound according to Formula I can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds according to Formula I may also be administered parenterally. Solutions or suspensions of the active compound can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably, a compound according to Formula I is administered orally.

The term “effective amount” is the quantity of compound in which a beneficial clinical outcome is achieved when the compound is administered to a subject. A “beneficial clinical outcome” includes amelioration or improvement of the clinical symptoms of the disease, disorder or syndrome, prevention, inhibition or a delay in the recurrence of symptom of the disease or of the disease itself and/or an increase in the longevity of the subject compared with the absence of the treatment, or prevention, inhibition or a delay in the progression of symptom of the disease or of the disease itself, such as, the progression of NASH to cirrhosis. The precise amount of compound (or other therapeutic agent) administered to a subject will depend on the type and severity of the disease or condition and on the characteristics of the subject, such as general health, age, sex, body weight and tolerance to drugs. It will also depend on the degree, severity and type of disorder. The skilled artisan will be able to determine appropriate dosages depending on these and other factors. When co-administered with another therapeutic agent, an “effective amount” of the second agent will depend on the type of drug used. The effective dosage may vary depending on the mode of administration.

A compound according to Formula I can be administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligram to about 50 milligrams. In the case of a 70 kg adult human, the total daily dose will generally be from about 7 milligrams to about 350 milligrams. This dosage regimen may be adjusted to provide the optimal therapeutic response.

The invention is illustrated by the following examples which are not intended to be limiting in any way.

Experimental

Compound A as used in the Examples and Figures is 1-(4-{4-[4-(4-Chloro-phenyl)-pyrimidin-2-ylamino]-benzoyl} -piperazin-1-yl)-ethanone.

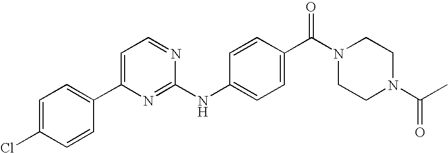

Animals, feeding and treatments Two groups of 8 week old male C56BL/6 mice were set under a high sucrose containing diet together with 1% Orotic acid (HSD) for 4 weeks (TD 02366, Tekland Mills) (Feldstein et al. J Hepatol, 39:978-983(2003)). All animals were purchased from Jackson Laboratories and were treated and handled following the National Academy of Sciences criteria (NIH publication 86-23 revised 1985). One group of animals received a daily subcutaneous injection of compound A at a concentration of 30 mg/kg (compound A) and the second group received vehicle with the same frequency (Vehicle). Additionally, a third group of mice was fed under a normal chow diet (control).

Serum analysis Blood was taken from mice after fasting and glucose levels were measured with Ascensia Elite sensor (Bayer). Serum Insulin (Linco Research) and Adiponectin (R&D systems) levels were quantified by ELISA.

Histological evaluation Livers from mice were harvested and after fixation with formaldehyde embedded in paraffin for further histological evaluation. Hematoxylin & Eosin staining was performed in liver sections and steatosis was assessed following a semiquantitative scoring system. Oil red 0 staining was performed to evidence the presence of intracellular lipds. Liver fibrosis was determined by Sirius Red which stains collagen fibres. Scoring of fibrosis was performed following the Brunt system. Additionally, immunohistochemistry (IHC) was performed on cryosections for the polyclonal anti-P65 (A) antibody, dilution 1/50 and anti-TNFα antibody, dilution 1/20 (both from Santa Cruz Biotechnology Inc.). IHC was performed on paraffin-embedded material for the Nitrotyrosine (C-3NT) antibody, dilution 1:50 (Upstate). For iNOS, the paraffin sections were pretreated in citrate buffer (pH 6.0) for 30 min in the hote water bath at 98.5° C.

Subsequently, the slides were incubated with anti-rabbit peroxidase-conjugated EnVision antibody (Dako Corp.) for 30 minutes at room temperature. The reaction product was developed with the use of 3-amino-9-ethylcarbazol and H₂O₂, 0.01% for cryostat sections and with the use of 3.3-diaminobenzidine tetrahdrochloride and H₂O₂, 0.01% for paraffin embedded sections. Negative controls consisted of omission of the primary antibody.

Western blot analysis Whole liver protein extracts were obtained by using ice- cold buffer (20 mM Tris pH 7.5, 150 mM NaCl, 1 mM EGTA, 1 mM EDTA, 1% Triton X-100, 2.5 mM sodium pyrophosphate and cocktail of protease inhibitors). 10% SDS-polyacrylamide-gel electrophoresis was performed for immunoblotting analysis. Membranes were probed with the following antibodies: l-κBα (#9162s) from Cell Signaling Technology, PPARα (ab8934) and PPARγ (ab19481) from AbCam and tubulin (10806) from Sigma. As secondary antibodies we used anti-rabbit-IgG-HRP-linked (#7074) from Cell Signaling Technology and anti-mouse-lgG-HRP-linked (sc-2005) from Santa Cruz Biotechnology.

RNA isolation and quantitative Real Time PCR RNA from frozen liver tissue was extracted with PeqGOLD-RNAPure kit (Peqlab). First strand synthesis was performed with Oligo dT primers and reverse transcription with M-MLV Reverse Transcriptase (Invitrogen Corp.) Quantitative Real Time PCR was performed with the AbbyPrism 7300 Real-Time PCR system from Applied Biosystems by using SYBR Green Reagent (Applied biosystems). Real time reactions were performed twice in triplicates. All values were normalized to the level of GAPDH. Primer sequences are detailed in Table 1.

Detection of apoptosis Apoptotic cell death was determined in liver tissue by using two difference strategies. Caspase-3 activity was analysed in whole liver extracts. Proteins were extracted with the lysis buffer containing 1M Hepes, 10% CHAPS, 0.5 EDTA, 1 m DTT and 0.1 M pefa-block and further incubated with the fluorescent substrate Ac-DEVD-AMC (Biomol). The enzymatic activity was then measured at λ. excitation 390 nm and λ emission 510 nm and the result was corrected with the protein concentration. Additionally, we performed a TUNEL assay on frozen liver sections using the “In situ cell death detection Kit, POD” (Roche Diagnostics Corp.) following manufacturer's instructions.

Statistical analysis Data are expressed as mean ± standard deviation of the mean (sdm). 2-ways analysis of variance followed by Student's T test was used in order to determine statistical significance.

### EXAMPLE 1

### Inhibition of High Sucrose Diet Induced Obesity using Compound A

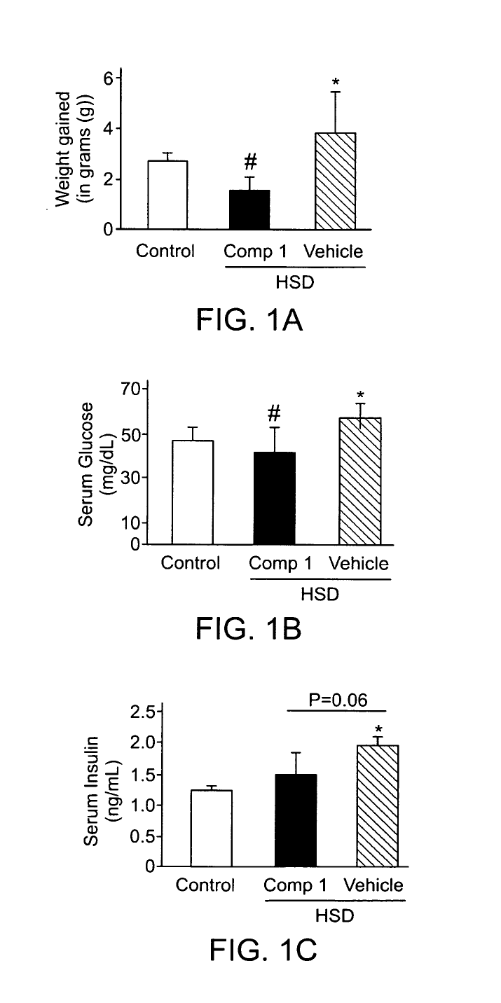

A significant weight gain was observed in vehicle treated mice after 4 weeks of HSD feeding compared to compound A treated mice and chow control animals (FIG. 1A). Vehicle treated mice exhibited higher fat accumulation in the abdominal cavity while animals that received compound A evidenced less amount of fat that was not significantly different compared to control chow mice which showed no macroscopical fat deposits.

### EXAMPLE 2

### Inhibition of High Sucrose Diet Induced Insulin Resistance using Compound A

Evidences of insulin resistance were observed in HSD/vehicle mice as blood glucose levels were significantly higher in this group compared to animals treated with either HSD/compound A or mice under chow diet (FIG. 1B). Accordingly, serum insulin levels were significantly higher in HSD/vehicle treated animals when compared to control animals while HSD/compound A mice exhibited roughly significantly less blood insulin that HSD/vehicle treated animals (p=0.06) (FIG. 1C).

### EXAMPLE 3

### Inhibition of High Sucrose Diet Induced NASH using Compound A

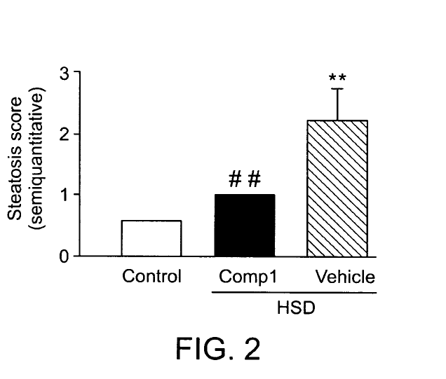

Haematoxylin and eosin (H&E) stainings on liver sections revealed profuse presence of fat droplets as well as caryorexis and apoptotic bodies in HSD/vehicle treated animals. Steatosis was numerically scored following semiquantitative pathological standards and was defined as micro- to mediovesicular steatosis (FIG. 2). HSD/compound A treated mice presented a liver histology similar to chow control animals with no clear evidence of fat accumulation (FIG. 2). Oil-Red-O staining confirmed the results of H&E analysis showing a high presence of fat deposits in livers from HSD/vehicle treated mice in the form of macro to mediovesicular steatosis while only minor microvesicular fat dropples could be observed in some of the HSD/compound A mice analysed. Chow diet fed mice exhibited a completely negative Oil-Red-O staining.

### EXAMPLE 4

### Compound A Attenuates the HSD Induced Inflammatory Response in the Liver

NF-κB is draped in the cytoplasm via I-κBα and its phosphorylation and further degradation results in nuclear translocation of NF-αB and target gene transcription. The I-κBα phosphor-status was studied in HSD (Treated) and chow diet fed (control) animals. Western blot analysis of whole liver extracts revealed lower levels of I-κBα in HSD/vehicle treated animals while I-κBα was clearly present in HSD/compound A and chow fed control animals. Accordingly, immunohistochemistry on liver sections using a p65 antibody showed strong NF-κB activation in non-parenchymal cells in HSD/vehicle mice while stainings were practically negative in HSD/compound A and control animals. Thus, HSD triggers NF-κB activation in vehicle treated animals while its induction is reduced in mice treated with compound A.

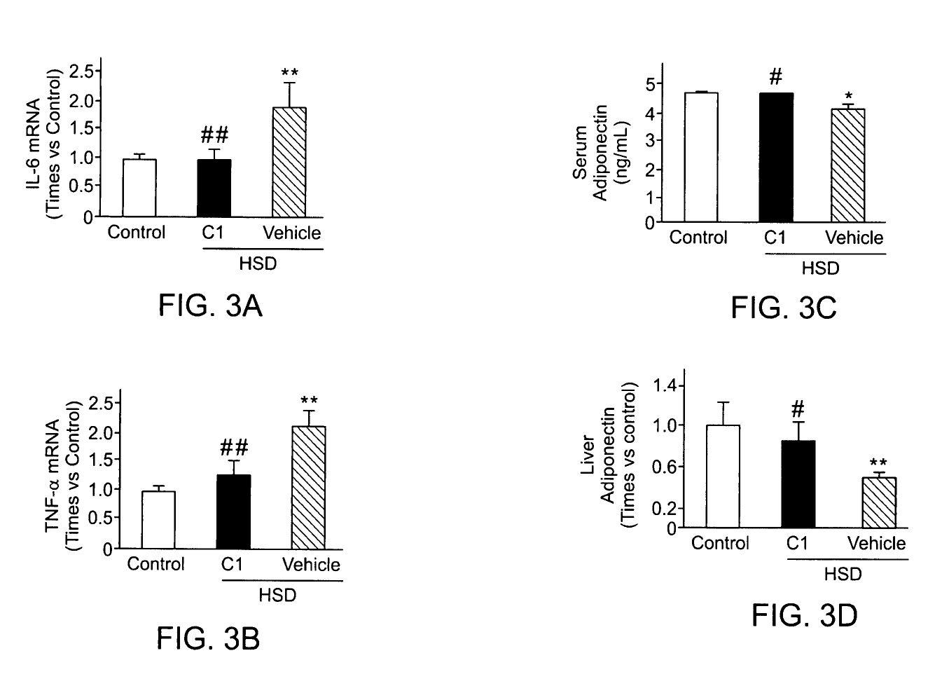

Steatosis sensitizes the liver to a variety of ‘second hits’ which will trigger necroinflammation and fibrosis. Inflammation and oxidative stress are essential factors involved in the progression of simple steatosis to hepatocellular injury and NASH (Farrell et al. Hepatology, 43:S99-S112 (2006)). The expression of IL-6 and TNF was studied by Real Time RT-PCR. In HSD/vehicle treated animals an upregulation of IL-6 and TNF mRNA expression was found compared to the HSD/compound A and the chow control group (FIG. 3A and 3B). Immunohistochemistry using a TNF antibody confirmed the higher presence of this pro-inflammatory cytokine in HSD/vehicle treated animals as evidenced by TNF positivity in non-parenchymal cells. These results demonstrate that HSD triggers liver inflammation and this response can be reverted by using compound A.

### EXAMPLE 5

### Compound A Attenuates the HSD Induced Lipid Accumulation in the Liver

NASH is associated with high TNF expression accompanied by downregulation of adiponectin levels (Musso et al. Hepatology, 42:1175-1183(2005)). Adiponectin is an adipokine with strong anti-lipogenic activity along with anti-inflammatory and anti-fibrogenic effects which negatively regulates TNF and protects the liver from lipid accumulation (Hui et al. Hepatology 40:46-54 (2004)) (Kamada et al. Gastroenterologyl, 125:1796-1807(2003)) (Maeda et al. Nat. Med, 8:731-737 (2002)). After 4 weeks of HSD feeding no significant change in adiponectin expression was found in the serum and liver from HSD/compound A treated animals compared to chow controls. However, a significant decrease in adiponectin serum and liver levels was observed in the HSD/vehicle group. Thus, adiponectin levels negatively correlates with the higher TNF expression found in these livers (FIG. 3C and 3D).

### EXAMPLE 6

### Compound A Improves Lipid Catabolism

During fasting or pathophysiological situations, like dietary-induced obesity and insulin resistance, free fatty acids (FFA) are released from the adipose tissue. Then, the liver uptakes circulating FFA which are transported into the mitochondria by CPT-1 for β-oxidation. Malonyl-CoA derive from Acetyl-CoA inhibits the CPT-1 mediated FFA transport into the mitochondria and facilitates its accumulation in the liver and further conversion in triglycerides (Pessayre et al. Am. J. Physiol. Gastrointest Liver Physiol, 282:G193-199(2002)). The nuclear receptor PPARα has anti-flammatory and anti-fibrogenic effects and is one of the master regulators of lipid metabolism. PPARa regulates transcription of CPT-1 and Acyl-CoA oxidase (ACOX) which enhance mitochondrial and peroxisomal α-oxidation thus limiting FFA accumulation in the liver (Aoyama et al. J Biol Chem, 273:5678-5684 (1998)) (Farrell et al. Hepatology, 38:123-132 (2003)) (Tugwood et al. Embo J, 11:433-439 (1992)) (Fan et al. J. Biol. Chem., 273:15639-15645 (1998)).

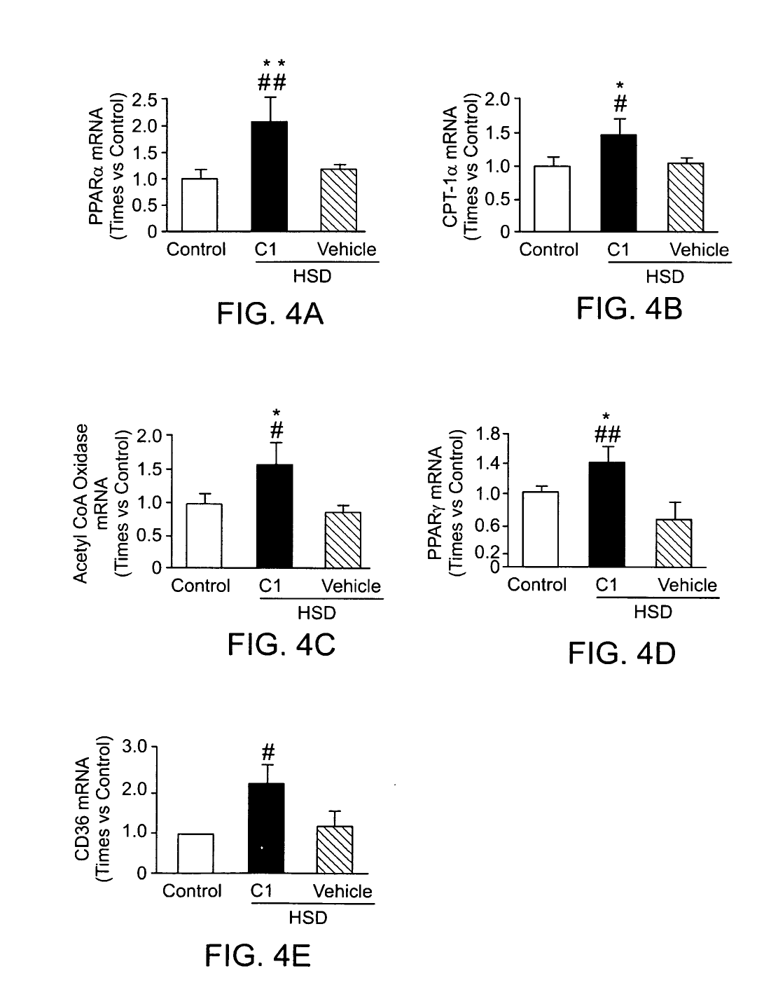

PPARα real time RT-PCR analysis revealed significantly higher expression in liver from HSD fed mice treated with compound A while no significant differences were found between the HSD/vehicle group and the chow control animals (FIG. 4A). Accordingly with the mRNA expression, western blot analysis showed a higher presence of PPARα in livers from HSD/compound A mice than in HSD/vehicle animals. Concomitantly, CPT-1 and ACOX were up-regulated in livers from mice that received the compound A compared to vehicle treated animals as shown by real time RT-PCR (FIG. 4B and 4C). Thus, these results indicate that PPARα expression and its target genes involved in lipid metabolism are clearly up-regulated in compound A-treated animals under HSD. Additionally, PPARγ was upregulated in livers derived from these animals, while it was significantly downregulated in vehicle injected animals (FIG. 4D). Western blot analysis clearly showed lower PPARγ protein expression in HSD/vehicle animals and a more pronounced presence in compound A mice. Additionally, CD36, the fatty acid translocase involved in the uptake of FFAs and liver insulin resistance (Aitman et al. Nat Genet, 21:76-83 (1999)) (Goudriann et al. J Lipid Res, 44:2270-2277 (2003)) is also upregulated in HSD/compound A mice as evidenced by real time RT-PCR (FIG. 4E). These data suggest that inhibition of NF-κB in compound A mice facilitates expression of both PPARs and as a consequence improves inflammation and lipid metabolism.

### EXAMPLE 7

### Compound A Ameliorates Oxidative Stress and ROS Formation in HSD Fed Mice

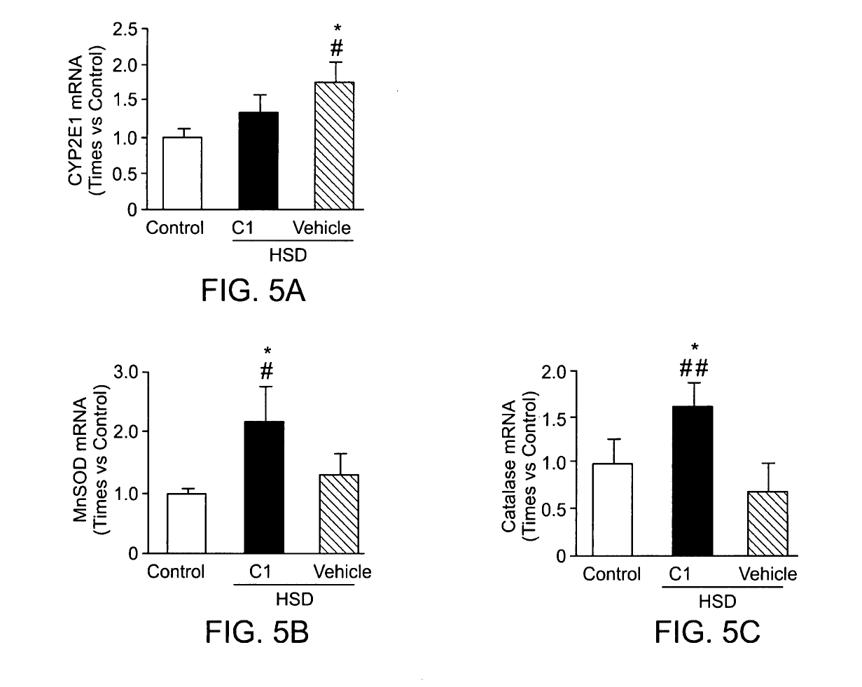

Besides inflammation, oxidative stress and ROS formation mediate progression from steatosis to NASH. Cytochrome P450 2E1 (CYP2E1) is elevated in both human disease and animal models of NASH and it is closely related to impaired hepatic insulin signaling (Schattenberg et al. J Biol Chem, 280:9887-9894 (2005)) (Weltman et al. Gastroenterology, 111:1645-1654 (1996)). A significant increase in CYP2E1 mRNA expression was found in HSD/vehicle animals compared to chow control mice. Compound A treatment reduced HSD induced upregulation of CYP2E1 and its expression was not significantly different than the chow control group (FIG. 5A).

CYP2E1 has an enhanced NAPDH oxidase activity which induces superoxide anion (O₂) and H₂O₂ formation (Ekstrom et al. Biocehm Pharmacol, 38:131301319 (1989)). ROS production is further increased via lipid peroxidation and both mechanisms together contribute to aggravation of NASH. Antioxidant enzymes such as SOD are downregulated in genetically obese (ob/ob) mice and antioxidant therapy has proved to have beneficial effects (Laurent et al. Hepatology, 39:1277-1285 (2004)). We thus studied mRNA expression of the antioxidant enzymes MnSOD and Catalase. Both enzymes were significantly higher in compound A treated animals compared to vehicle treated mice and chow controls (FIG. 5B). Oxidative stress and inflammation induces genes like iNOS and thus NO production. iNOS and NO trigger the production of metabolites such as peroxynitrites (Jaeschke et al. Am J Physiol Gastroenterol Liver Physiol, 284:G15-26 (2003)). Immunohistochemistry with a 3-NytroTirosine antibody showed high incidence of peroxynitrites in livers from HSD/vehicle treated animals localized in the centrolobular area (FIG. 5C).

### EXAMPLE 8

### Compound A Prevents Progression of NASH by Attenuating Hepatocyte Apoptosis and Liver Fibrosis

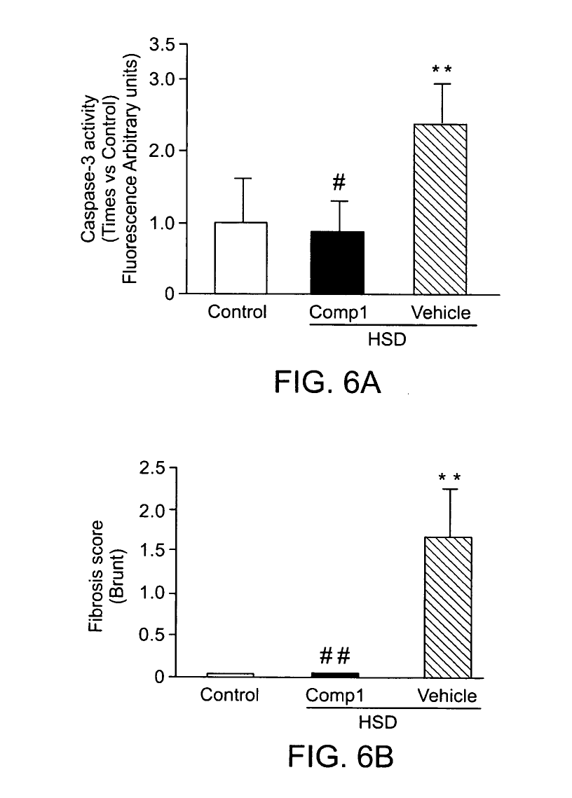

Apoptosis is increased in patients with NASH and has a direct impact on disease activity and the degree of hepatic fibrosis (Canbay et al. Heptaology 39:273-278 (2004)). Caspase-3 activity was measured in order to determine the rate of cell death in the three groups. HSD resulted in a 2.5 fold increase in caspase 3 activity compared to chow control mice. Compound A treatment strongly reduced caspase 3 activity to a level as found in chow control mice (FIG. 6A). These results were further confirmed by TUNEL assays. HSD/vehicle feeding induced an increase in TUNEL-positive cells, which could be blocked using compound A.

Sirius red staining on liver sections was performed to visualize liver collagen expression. HSD/vehicle animals evidenced accumulation and fine thickening of reticules fibers around the centrolobular areas. In contrast, no significant degree of collagen expression could be detected in HSD/compound A treated animals and in control mice (FIG. 6B). Fibrosis was quantified and scored according to Brunt score (Brunt et al. Semin Liver Dis, 21:3-16 (2001)). A score of 1.6 was found for HSD/vehicle treated animals, while the level was virtually zero in HSD/compound A and chow fed animals. Therefore our results demonstrate that compound A reduces NASH progression by blocking the degree of hepatocyte apoptosis and as a consequence the amount of collagen deposition.

While this invention has been particularly shown and described with references to example embodiments thereof, it will be understood by those skilled in the art that various changes in form and details may be made therein without departing from the scope of the invention encompassed by the appended claims.

1. A method of treating a subject with nonalcoholic fatty liver disease (NAFLD)/non-alcoholic steatohepatitis (NASH) comprising administering to the subject an effective amount of a compound according to Formula I: 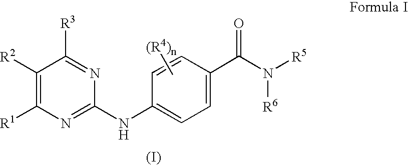
or a pharmaceutically acceptable salt thereof, wherein:
R¹ is either aryl or heteroaryl optionally substituted with one to four substituents independently selected from R⁷;
R² is hydrogen;
R³ is either hydrogen or lower alkyl;
R⁴ is, in each instance, independently selected from the group consisting of halogen, hydroxy, lower alkyl and lower alkoxy; wherein n is an integer from 0 to 4;
R⁵ and R⁶ are the same or different and are independently selected from the group consisting of —R⁸, —(CH₂)ₐC(═O)R⁹, —(CH₂)ₐC(═O)OR⁹, —(CH₂)ₐC(═O)NR⁹R¹⁰, (CH₂)ₐC(═O)NR⁹(CH₂)_{b}C(═O)R¹⁰, —(CH₂)ₐNR¹¹C(═O)NR⁹R¹⁰, —(CH₂)ₐNR⁹R¹⁰, —(CH₂)ₐOR⁹, —(CH₂)ₐNR⁹C(═O)R¹⁰, —(CH₂)ₐSO_{c}R⁹ and —(CH₂)ₐSO₂NR⁹R¹⁰; or
R⁵ and R⁶ taken together with the nitrogen atom to which they are attached to form an optionally substituted heterocycle;
R⁷is at each occurrence independently selected from the group consisting of halogen, hydroxy, cyano, nitro, carboxy, alkyl, alkoxy, haloalkyl, acyloxy, sulfanylalkyl, sulfinylalkyl, sulfonylalkyl, hydroxyalkyl, aryl, substituted aryl, alkylaryl, substituted alkylaryl, heterocycloalkyl, substituted heterocycloalkyl, alkylheterocycloalkyl, substituted alkylheterocycloalkyl, —C(═O)OR⁸, —OC(═O)R⁸, —C(═O)NR⁸R⁹, —C(═O)NR⁸OR⁹, —SO_{c}R⁸, —SO_{c}NR⁸R⁹, —NR⁸SO_{c}R⁹—NR⁸R⁹, —NR⁸C(═O)R⁹, —NR⁸C(═O)(CH₂)_{b}OR⁹, —NR⁸C(═O)(CH₂)_{b}R⁹, —O(CH₂)_{b}NR⁸R⁹ and heterocycloalkyl fused to phenyl;
R⁸, R⁹, R¹⁰ and R¹¹ are the same or different and are at each occurrence independently selected from the group consisting of hydrogen, alkyl, substituted alkyl, aryl, substituted aryl, alkylaryl, substituted alkylaryl, heterocycloalkyl, substituted heterocycloalkyl, alkylheterocycloalkyl and substituted alkylheterocycloalkyl; or
R⁸ and R⁹ taken together with the atom or atoms to which they are attached form an optionally substituted heterocycle;
a and b are the same or different and are at each occurrence independently selected from the group consisting of 0, 1, 2, 3 and 4; and
c is at each occurrence 0, 1 or 2.

2. The method according to claim 1, wherein R⁵ and R⁶, taken together with the nitrogen atom to which they are attached form an optionally substituted nitrogen- containing non-aromatic heterocycle.

3. The method according to claim 2, wherein the nitrogen-containing non-aromatic heterocycle is selected from the group consisting of morpholinyl, thiomorpholinyl, pyrrolidinonyl, pyrrolidinyl, piperidinyl, homopiperidinyl, piperazinyl, homopiperazinyl, hydantoinyl, tetrahydropyridinyl, tetrahydropyrimidinyl, oxazolidinyl, thiazolidinyl, indolinyl, isoindolinyl, tetrahydroquinolinyl and tetrahydroisoquinolinyl.

4. The method according to claim 1, wherein R¹ is either aryl or heteroaryl.

5. The method according to claim 1, wherein R¹ is selected from the group consisting of aryl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, 25 pyrazolyl, isothiazolyl, pyridazinyl, pyrimidinyl, pyrazinyl, triazinyl, cinnolinyl, phthalazinyl and quinazolinyl.

6. The method according to claim 1, wherein R¹ is phenyl.

7. The method according to claim 3, wherein the nitrogen-containing heterocycle is piperazinyl.

8. The method according to claim 3, wherein the nitrogen-containing heterocycle is piperidinyl.

9. The method according to claim 3, wherein the nitrogen-containing heterocycle is morpholinyl.

10. The method according to claim 1, wherein said compound for the treatment of non-alcoholic fatty liver disease (NAFLD)/non-alcoholic steatohepatitis is a compound according to Formula (II): 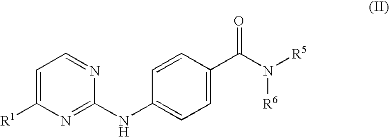
or a pharmaceutically acceptable salt thereof, wherein
R¹ s aryl or heteroaryl optionally substituted with one to four substituents independently selected from R⁷;
R⁵ and R⁶ are the same or different and are independently selected from the group consisting of —R⁸, —(CH₂)ₐC(═O)R⁹, —(CH₂)ₐC(═O)OR⁹, —(CH₂)ₐC(═O)NR⁹R¹⁰, —(CH₂)ₐC(═O)NR⁹(CH₂)_{b}C(═O)R¹⁰, —(CH₂)ₐSO_{c}R⁹, —(CH₂)ₐNR⁹C(═O)R¹⁰, —(CH₂)ₐNR¹¹C(═O)NR⁹R¹⁰, —(CH₂)ₐNR⁹R¹⁰, (CH₂)ₐOR⁹ and —(CH₂)ₐSO₂NR⁹R¹⁰;
or R⁵ and R⁶ taken together with the nitrogen atom to which they are attached form a heterocycle or substituted heterocycle;
R⁷is at each occurrence independently selected from the group consisting of halogen, hydroxy, cyano, nitro, carboxy, alkyl, alkoxy, haloalkyl, acyloxy, sulfanylalkyl, sulfinylalkyl, sulfonylalkyl, hydroxyalkyl, aryl, substituted aryl, alkylaryl, substituted alkylaryl, heterocycloalkyl, substituted heterocycloalkyl, alkylheterocycloalkyl, substituted alkylheterocycloalkyl, —C(═O)OR⁸, —NR⁸R⁹, —OC(═O)R⁸, —C(═O)NR⁸R⁹, —C(═O)NR⁸OR⁹, —SO_{c}R⁸, —SO_{c}NR⁸R⁹, —NR⁸SO_{c}R⁹, —NR⁸C(═O)R⁹, —NR⁸C(═O)(CH₂)_{b}OR⁹, —NR⁸C(═O)(CH₂)_{b}R⁹, —O(CH₂)_{b}NR⁸R⁹ and heterocycloalkyl fused to phenyl;
R⁸, R⁹, R¹⁰ and R¹¹ are the same or different and are at each occurrence independently selected from the group consisting of hydrogen, alkyl, substituted alkyl, aryl, substituted aryl, alkylaryl, substituted alkylaryl, heterocycloalkyl, substituted heterocycloalkyl, alkylheterocycloalkyl and substituted alkylheterocycloalkyl;
or R⁸ and R⁹ taken together with the atom or atoms to which they are attached form an optionally substituted heterocycle;
a and b are the same or different and are at each occurrence independently selected from the group consisting of 0, 1, 2, 3 and 4; and
c is at each occurrence 0, 1 or 2.

11. The method according to claim 10, wherein R⁵ and R⁶, taken together with the nitrogen atom to which they are attached form an optionally substituted nitrogen-containing non-aromatic heterocycle.

12. The method according to claim 11, wherein the nitrogen-containing non-aromatic heterocycle is selected from the group consisting of morpholinyl, thiomorpholinyl, pyrrolidinonyl, pyrrolidinyl, piperidinyl, homopiperidinyl, piperazinyl, homopiperazinyl, hydantoinyl, tetrahydropyridinyl, tetrahydropyrimidinyl, oxazolidinyl, thiazolidinyl, indolinyl, isoindolinyl, tetrahydroquinolinyl and tetrahydroisoquinolinyl.

13. The method according to claim 10, wherein R¹ is either aryl or heteroaryl.

14. The method according to claim 10, wherein R¹ is selected from the group consisting of aryl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl and quinazolinyl.

15. The method according to claim 10, wherein R¹ is phenyl.

16. The method according to claim 11, wherein the nitrogen-containing heterocycle is piperazinyl.

17. The method according to claim 11, wherein the nitrogen-containing heterocycle is piperidinyl.

18. The method according to claim 11, wherein the nitrogen-containing heterocycle is morpholinyl.

19. The method according to claim 10, wherein said compound effective for the treatment of non-alcoholic fatty liver disease (NAFLD)/non-alcoholic steatohepatitis (NASH) is a compound according to Formula (III): 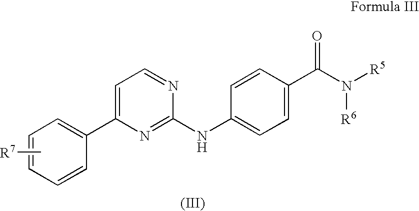
or a pharmaceutically acceptable salt thereof.

20. The method according to claim 10, wherein said compound effective for the treatment of non-alcoholic fatty liver disease (NAFLD)/non-alcoholic steatohepatitis (NASH) is a compound according to Formula (IV): 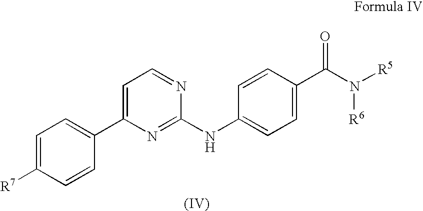
or a pharmaceutically acceptable salt thereof.

21. A method of treating a subject suffering from nonalcoholic fatty liver disease (NAFLD)/non-alcoholic steatohepatitis (NASH) comprising administering to the subject an effective amount of:1-(4-{4-[4-(4-Chloro-phenyl)-pyrimidin-2-ylamino]-benzoyl}-piperazin-1-yl)-ethanone, which is represented by the structural formula: 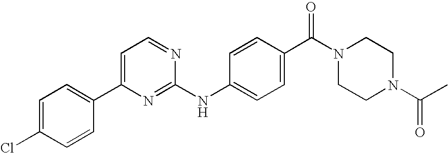
or a pharmaceutically acceptable salt thereof.

22. The method of claim 1, wherein the NAFLD/NASH is non-alcoholic-steatohepatitis.

23. The method of claim 1, wherein the NAFLD/NASH is fatty liver (steatosis).

24. The method of claim 1, wherein the NAFLD/NASH is cirrhosis.

25. The method of claim 1, further comprising administering to the subject an effective amount of a therapeutic agent selected from the group consisting of an agent used to lower blood glucose, an agent used to control lipid levels, an antioxidant, and an anti-inflammatory agent.

26. The method of claim 25, wherein the agent is selected from the group consisting of rosiglitazone, pioglitazone, metformin, ursodeoxycholic acid, selenium, betaine, vitamin E, clofibrate and gemfibrozil.

27. A method of treating insulin resistance in a subject, comprising administering to the subject an effective amount of a compound according to Formula I, provided that the subject is suffering from a disorder other than type II diabetes: 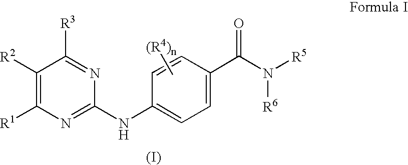
or a physiologically pharmaceutically acceptable salt thereof, wherein:
R¹ is either aryl or heteroaryl optionally substituted with one to four substituents independently selected from R⁷;
R² is hydrogen;
R³ is either hydrogen or lower alkyl;
R⁴ is, in each instance, independently selected from the group consisting of halogen, hydroxy, lower alkyl and lower alkoxy; wherein n is an integer from 0 to 4;
R⁵ and R⁶ are the same or different and are independently selected from the group consisting of —R⁸, —(CH₂)ₐC(═O)R⁹, —(CH₂)ₐC(═O)OR⁹, —(CH₂)ₐC(═O)NR⁹R¹⁰, —(CH₂)ₐC(═O)NR⁹(CH₂)_{b}C(═O)R¹⁰, —(CH₂)ₐNR¹¹C(═O)NR⁹R¹⁰, —(CH₂)ₐNR⁹R¹⁰, —(CH₂)ₐOR⁹, (CH₂)ₐNR⁹C(═O)R¹⁰, —(CH₂)ₐSO_{c}R⁹ and —(CH₂)ₐSO₂NR⁹R¹⁰; or R⁵ and R⁶ taken together with the nitrogen atom to which they are attached to form an optionally substituted heterocycle;
R⁷ is at each occurrence independently selected from the group consisting of halogen, hydroxy, cyano, nitro, carboxy, alkyl, alkoxy, haloalkyl, acyloxy, sulfanylalkyl, sulfinylalkyl, sulfonylalkyl, hydroxyalkyl, aryl, substituted aryl, alkylaryl, substituted alkylaryl, heterocycloalkyl, substituted heterocycloalkyl, alkylheterocycloalkyl, substituted alkylheterocycloalkyl, —C(═O)OR⁸, —OC(═O)R⁸, —C(═O)NR⁸R⁹, —C(═O)NR⁸OR⁹, —SO_{c}R⁸, —SO_{c}NR⁸R⁹, —NR⁸SO_{c}R⁹—NR⁸R⁹, —NR⁸C(═O)R⁹, —NR⁸C(═O)(CH₂)_{b}OR⁹, —NR⁸C(═O)(CH₂)_{b}R⁹, —O(CH₂)_{b}NR⁸R⁹ and heterocycloalkyl fused to phenyl;
R⁸, R⁹, R¹⁰ and R¹¹ are the same or different and are at each occurrence independently selected from the group consisting of hydrogen, alkyl, substituted alkyl, aryl, substituted aryl, alkylaryl, substituted alkylaryl, heterocycloalkyl, substituted heterocycloalkyl, alkylheterocycloalkyl and substituted alkylheterocycloalkyl; or
R⁸ and R⁹ taken together with the atom or atoms to which they are attached form an optionally substituted heterocycle;
a and b are the same or different and are at each occurrence independently selected from the group consisting of 0, 1, 2, 3 and 4; and
c is at each occurrence 0, 1 or 2.

28-56. (canceled)

57. The method of treating obesity in a subject, comprising administering to the subject an effective amount of a compound according to Formula I: 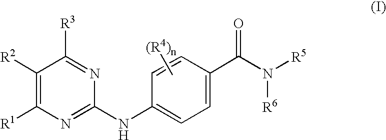
or a pharmaceutically acceptable salts thereof, wherein
R¹ is either aryl or heteroaryl optionally substituted with one to four substituents independently selected from R⁷; R² is hydrogen;
R³ is either hydrogen or lower alkyl;
R⁴ is, in each instance, independently selected from the group consisting of halogen, hydroxy, lower alkyl and lower alkoxy; wherein n is an integer from 0 to 4;
R⁵ and R⁶ are the same or different and are independently selected from the group consisting of—R⁸, —(CH₂)ₐC(═O)R⁹, —(CH₂)ₐC(═O)OR⁹, (CH₂)ₐC(═O)NR⁹R¹⁰, —(CH₂)ₐC(═O)NR⁹(CH₂)_{b}C(═O)R¹⁰, —(CH₂)ₐNR¹¹C(═O)NR⁹R¹⁰, —(CH₂)ₐNR⁹R¹⁰, —(CH₂)ₐOR⁹, —(CH₂)ₐNR⁹C(═O)R¹⁰, —(CH₂)ₐSO_{c}R⁹ and —(CH₂)ₐSO₂NR⁹R¹⁰;
or R⁵ and R⁶ taken together with the nitrogen atom to which they are attached to form an optionally substituted heterocycle;
R⁷is at each occurrence independently selected from the group consisting of halogen, hydroxy, cyano, nitro, carboxy, alkyl, alkoxy, haloalkyl, acyloxy, sulfanylalkyl, sulfinylalkyl, sulfonylalkyl, hydroxyalkyl, aryl, substituted aryl, alkylaryl, substituted alkylaryl, heterocycloalkyl, substituted heterocycloalkyl, alkylheterocycloalkyl, substituted alkylheterocycloalkyl, —C(═O)OR⁸, —OC(═O)R⁸, —C(═O)NR⁸R⁹, —C(═O)NR⁸OR⁹, —SO_{C}R⁸, —SO_{C}NR⁸R⁹, —NR⁸SO_{C}R⁹—NR⁸R⁹, —NR⁸C(═O)R⁹, —NR⁸C(═O)(CH₂)_{b}OR⁹, —NR⁸C(═O)(CH₂)_{b}R⁹, —O(CH₂)_{b}NR⁸R⁹ and heterocycloalkyl fused to phenyl;
R⁸, R⁹, R¹⁰ and R¹¹ are the same or different and are at each occurrence independently selected from the group consisting of hydrogen, alkyl, substituted alkyl, aryl, substituted aryl, alkylaryl, substituted alkylaryl, heterocycloalkyl, substituted heterocycloalkyl, alkylheterocycloalkyl and substituted alkylheterocycloalkyl;
or R⁸ and R⁹ taken together with the atom or atoms to which they are attached form an optionally substituted heterocycle;
a and b are the same or different and are at each occurrence independently selected from the group consisting of 0, 1, 2, 3 and 4; and
c is at each occurrence 0, 1 or 2.

58-79. (canceled)

80. A method of treating hyperlipidemia in a subject, comprising administering to the subject an effective amount of a compound according to Formula I: 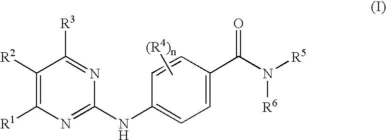
or a pharmaceutically acceptable salts thereof, wherein
R¹ is either aryl or heteroaryl optionally substituted with one to four substituents independently selected from R⁷;
R² is hydrogen;
R³ is either hydrogen or lower alkyl;
R⁴ is, in each instance, independently selected from the group consisting of halogen, hydroxy, lower alkyl and lower alkoxy; wherein n is an integer from 0 to 4;
R⁵ and R⁶ are the same or different and are independently selected from the group consisting of —R⁸, —(CH₂)ₐC(═O)R⁹, —(CH₂)ₐC(═O)OR⁹, —(CH₂)ₐC(═O)NR⁹R¹⁰, —(CH₂)ₐC(═O)NR⁹(CH₂)_{b}C(═O)R¹⁰, —(CH₂)ₐNR¹¹C(═O)NR⁹R¹⁰,—(CH₂)ₐNR⁹R¹⁰, —(CH₂)ₐOR⁹, —(CH₂)ₐNR⁹C(═O)R¹⁰, —(CH₂)ₐSO_{c}R⁹ and —(CH₂)ₐSO₂NR⁹R¹⁰; or
R⁵ and R⁶ taken together with the nitrogen atom to which they are attached to form an optionally substituted heterocycle;
R⁷ is at each occurrence independently selected from the group consisting of halogen, hydroxy, cyano, nitro, carboxy, alkyl, alkoxy, haloalkyl, acyloxy, sulfanylalkyl, sulfinylalkyl, sulfonylalkyl, hydroxyalkyl, aryl, substituted aryl, alkylaryl, substituted alkylaryl, heterocycloalkyl, substituted heterocycloalkyl, alkylheterocycloalkyl, substituted alkylheterocycloalkyl, —C(═O)OR⁸, —OC(═O)R⁸, —C(═O)NR⁸R⁹, —C(═O)NR⁸OR⁹, —SO_{C}R⁸, —SO_{C}NR⁸R⁹, —NR⁸SO_{C}R⁹—NR⁸R⁹, —NR⁸C(═O)R⁹, —NR⁸C(═O)(CH₂)_{b}OR⁹, —NR⁸C(═O)(CH₂)_{b}R⁹, —O(CH₂)_{b}NR⁸R⁹ and heterocycloalkyl fused to phenyl;
R⁸, R⁹, R¹⁰ and R¹¹ are the same or different and are at each occurrence independently selected from the group consisting of hydrogen, alkyl, substituted alkyl, aryl, substituted aryl, alkylaryl, substituted alkylaryl, heterocycloalkyl, substituted heterocycloalkyl, alkylheterocycloalkyl and substituted alkylheterocycloalkyl; or
R⁸ and R⁹ taken together with the atom or atoms to which they areattached form an optionally substituted heterocycle;
a and b are the same or different and are at each occurrence independently selected from the group consisting of 0, 1, 2, 3 and 4; and
c is at each occurrence 0, 1 or 2.

81-103. (canceled)

104. A method of treating a subject with alcoholic steatohepatitis comprising administering to the subject an effective amount of a compound according to Formula I: 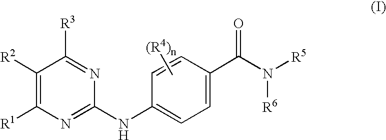
or a pharmaceutically acceptable salts thereof, wherein
R¹ is either aryl or heteroaryl optionally substituted with one to four substituents independently selected from R⁷;
R² is hydrogen;
R³ is either hydrogen or lower alkyl;
R⁴ is, in each instance, independently selected from the group consisting of halogen, hydroxy, lower alkyl and lower alkoxy; wherein n is an integer from 0 to 4;
R⁵ and R⁶ are the same or different and are independently selected from the group consisting of —R⁸, —(CH₂)ₐC(═O)R⁹, —(CH₂)ₐC(═O)OR⁹, —(CH₂)ₐC(═O)NR⁹R¹⁰, —(CH₂)ₐC(═O)NR⁹(CH₂)_{b}C(═O)R¹⁰, —(CH₂)ₐNR¹¹C(═O)NR⁹R¹⁰, —(CH₂)ₐNR⁹R¹⁰, —(CH₂)ₐOR⁹, —(CH₂)ₐNR⁹C(═O)R¹⁰, —(CH₂)ₐSO_{c}R⁹ and —(CH₂)ₐSO₂NR⁹R¹⁰; or
R⁵ and R⁶ taken together with the nitrogen atom to which they are attached to form an optionally substituted heterocycle;
R⁷ is at each occurrence independently selected from the group consisting of halogen, hydroxy, cyano, nitro, carboxy, alkyl, alkoxy, haloalkyl, acyloxy, sulfanylalkyl, sulfinylalkyl, sulfonylalkyl, hydroxyalkyl, aryl, substituted aryl, alkylaryl, substituted alkylaryl, heterocycloalkyl, substituted heterocycloalkyl, alkylheterocycloalkyl, substituted alkylheterocycloalkyl, —C(═O)OR⁸, —OC(═O)R⁸, —C(═O)NR⁸R⁹, —C(═O)NR⁸OR⁹, —SO_{C}R⁸, —SO_{C}NR⁸R⁹, —NR⁸SO_{C}R⁹—NR⁸R⁹, —NR⁸C(═O)R⁹, —NR⁸C(═O)(CH₂)_{b}OR⁹, —NR⁸C(═O)(CH₂)_{b}R⁹, —O(CH₂)_{b}NR⁸R⁹ and heterocycloalkyl fused to phenyl;
R⁸, R⁹, R¹⁰ and R¹¹ are the same or different and are at each occurrence independently selected from the group consisting of hydrogen, alkyl, substituted alkyl, aryl, substituted aryl, alkylaryl, substituted alkylaryl, heterocycloalkyl, substituted heterocycloalkyl, alkylheterocycloalkyl and substituted alkylheterocycloalkyl; or
R⁸ and R⁹ taken together with the atom or atoms to which they are attached form an optionally substituted heterocycle;
a and b are the same or different and are at each occurrence independently selected from the group consisting of 0, 1, 2, 3 and 4; and
c is at each occurrence 0, 1 or 2.

105-125. (canceled)

126. A method of treating a subject with acute liver failure comprising administering to the subject an effective amount of a compound according to Formula I: 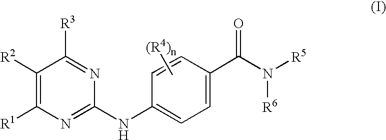
or a pharmaceutically acceptable salts thereof, wherein
R¹ is either aryl or heteroaryl optionally substituted with one to four substituents independently selected from R⁷;
R² is hydrogen;
R³ is either hydrogen or lower alkyl;
R⁴ is, in each instance, independently selected from the group consisting of halogen, hydroxy, lower alkyl and lower alkoxy; wherein n is an integer from 0 to 4;
R⁵ and R⁶ are the same or different and are independently selected from the group consisting of —R⁸, —(CH₂)ₐC(═O)R⁹, —(CH₂)ₐC(═O)OR⁹, —(CH₂)ₐC(═O)NR⁹R¹⁰, —(CH₂)ₐC(═O)NR⁹(CH₂)_{b}C(═O)R¹⁰, —(CH₂)ₐNR¹¹C(═O)NR⁹R¹⁰, —(CH₂)ₐNR⁹R¹⁰, —(CH₂)ₐOR⁹, —(CH₂)ₐNR⁹C(═O)R¹⁰, —(CH₂)ₐSO_{c}R⁹ and —(CH₂)ₐSO₂NR⁹R¹⁰; or
R⁵ and R⁶ taken together with the nitrogen atom to which they are attached to form an optionally substituted heterocycle;
R⁷ is at each occurrence independently selected from the group consisting of halogen, hydroxy, cyano, nitro, carboxy, alkyl, alkoxy, haloalkyl, acyloxy, sulfanylalkyl, sulfinylalkyl, sulfonylalkyl, hydroxyalkyl, aryl, substituted aryl, alkylaryl, substituted alkylaryl, heterocycloalkyl, substituted heterocycloalkyl, alkylheterocycloalkyl, substituted alkylheterocycloalkyl, —C(═O)OR⁸, —OC(═O)R⁸, —C(═O)NR⁸R⁹, —C(═O)NR⁸OR⁹, —SO_{C}R⁸, —SO_{C}NR⁸R⁹, —NR⁸SO_{C}R⁹—NR⁸R⁹, —NR⁸C(═O)R⁹, —NR⁸C(═O)(CH₂)_{b}OR⁹, —NR⁸C(═O)(CH₂)_{b}R⁹, —O(CH₂)_{b}NR⁸R⁹ and heterocycloalkyl fused to phenyl;
R⁸, R⁹, R¹⁰ and R¹¹ are the same or different and are at each occurrence independently selected from the group consisting of hydrogen, alkyl, substituted alkyl, aryl, substituted aryl, alkylaryl, substituted alkylaryl, heterocycloalkyl, substituted heterocycloalkyl, alkylheterocycloalkyl and substituted alkylheterocycloalkyl; or
R⁸ and R⁹ taken together with the atom or atoms to which they are attached form an optionally substituted heterocycle;
a and b are the same or different and are at each occurrence independently selected from the group consisting of 0, 1, 2, 3 and 4 ; and
c is at each occurrence 0, 1 or 2.

127-146. (canceled)
